(19) United States
(12) Patent Application Publication
(10) Pub. No.: US 2012/0232497 A1
(43) Pub. Date: Sep. 13, 2012

(54) PINCH CLAMP

(76) Inventor: Sukhvinder Singh, Wattle Grove (AU)

(21) Appl. No.: 13/498,025

(22) PCT Filed: Sep. 21, 2010
(86) PCT No.: PCT/AU2010/001229

§ 371 Date: May 29, 2012

(30) Foreign Application Priority Data
Sep. 23, 2009 (AU) 2009904606

(51) Int. Cl. A61M 39/28

(52) U.S. Cl. 604/250

(57) ABSTRACT

A non-reopening clamp including two arms hingedly connected, each arm having a clamping portion such that relative rotation of the arms brings the clamping portions into clamping engagement, wherein the arms include a locking mechanism for locking the clamping portions together. Access to the locking mechanism may be prevented by a shield, to prevent a user from re-opening the clamp.

### FIELD OF THE INVENTION

The present invention relates to clamps for flexible tubing. The invention has been developed primarily as a pinch clamp for use in the medical industry in procedures such as the collection of blood and will be described hereinafter with reference to this application. However, it will be appreciated that the invention is not limited to this particular field and can be used to clamp flexible tubing in any suitable environment.

### BACKGROUND OF THE INVENTION

In this specification unless the contrary is expressly stated, where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Pinch clamps are often used to control the flow of liquid in the collection of blood from donors or when handling blood and other IV liquids. These clamps are typically cheap and disposable. In some environments, to prevent contamination, spillage or for other reasons such as to comply with regulations, best practice guidelines and the like, there is a requirement that the clamps cannot be easily re-opened once closed.

In many instances, prior art clamps are difficult for a user to lock closed and some may protrude so as to be likely to tear or puncture surgical gloves, poke into the user's thumb or finger or snag to medical dressings, clothing or other fabric. Some prior art clamps do not provide auditory and/or visual confirmation of properly closure which may result in leakage and spillage of IV liquids or blood.

Advantageously the preferred embodiment of the present invention overcomes or ameliorates at least one of the disadvantages of the prior art or provides a useful alternative.

### SUMMARY OF THE INVENTION

According to a first aspect of the invention there is provided a clamp including:

two arms hingedly connected;
each arm having a clamping portion such that relative rotation of the arms brings the clamping portions into clamping engagement, wherein one of the arms includes a plurality of locking tabs and the other arm includes a plurality of receiving apertures for receiving the locking tabs, the locking tabs moving into the receiving apertures when the clamping portions come into clamping engagement thereby locking the clamp.

Preferably the arms are hingedly connected at one end, the other end of each arm having a handle adapted for finger engagement.

Preferably the clamping portion of each arm is located intermediate the ends of each respective arm.

Preferably the clamp includes a lock to keep the clamping portions in clamping engagement.

Preferably the lock is a non-reopening lock.

Preferably the lock is located intermediate the ends of each arm.

Preferably the locking tab extends substantially perpendicular to the direction of relative rotation of the arms.

Preferably the one arm includes two locking tabs each locking tab extending substantially perpendicular to the direction of relative rotation of the arms and the other arm includes two receiving apertures for receiving a respective locking tab.

Preferably the locking tabs and the receiving apertures are adapted for permanent engagement once the receiving apertures receive the locking tabs thereby forming the non-reopening lock.

Preferably the other arm includes means for preventing finger engagement of the locking tabs thereby preventing a user from opening the clamp. Preferably the other arm includes a shield extending substantially perpendicular to the direction of relative rotation of the arms, the shield having a hole therein to form the receiving aperture.

Preferably the other arm includes two shields extending substantially perpendicular to the direction of relative rotation of the arms, each shield having a hole therein to form the receiving aperture. Preferably the shields also act as a guide for tubing.

Preferably the shields are adapted to prevent finger engagement by a user thereby substantially preventing the user from unlocking the clamp. Preferably the arm also includes a stopper to prevent the user from unlocking the clamp.

Preferably the clamp includes an aperture through which a flexible length of tubing is guided.

Preferably the clamp includes means for preventing the clamp from sliding freely along the length of tubing.

According to a second aspect of the invention there is provided a clamp including:

two arms hingedly connected;
each arm having a clamping portion such that relative rotation of the arms brings the clamping portions into clamping engagement;
one of the arms having a locking tooth and the other arm having a receiving aperture for receiving the locking tooth, the locking tooth moving into locking engagement with the receiving aperture when the clamping portions come into clamping engagement thereby locking the clamp.

Preferably the arms are hingedly connected at their proximal end.

Preferably the locking tooth is located at the distal end of the one arm and the receiving aperture is located at the distal end of the other arm.

Preferably the clamping portions are located intermediate the ends of each respective arm.

Preferably the clamp has a clamping zone in which objects to be clamped are clamped when the clamping portions are brought into clamping engagement.

Preferably the clamping zone is adapted to receive a length of flexible tubing.

Preferably the clamping zone is adapted to received a length of flexible tubing, the length of flexible tubing running substantially perpendicular to the length of the clamp.

Preferably the clamping portions are suitably shaped to facilitate effective clamping of the tubing.

Preferably one of the clamping portions is triangular shaped and the other clamping portion is adapted to receive the triangular shaped clamping portion.

Preferably the locking tooth is connected at one end to the distal end of the one arm and has an abutment locking tab at its other end.

Preferably the receiving aperture is adapted to receive the abutment locking tab to thereby lock the clamp in clamping engagement.

Preferably the locking tooth is adapted to be permanently engagable with the receiving aperture such that the clamp is substantially a non-reopening clamp.

Preferably the arms are adapted for finger engagement by a user.

Preferably the clamp is a non-reopening clamp.

### BRIEF DESCRIPTION OF THE DRAWINGS

A preferred embodiment of the invention will now be described, by way of example only, with reference to the accompanying drawings in which:

FIG. 1 is a side view of a clamp according to the invention;

FIG. 2 is a perspective view of the clamp of FIG. 1;

FIG. 3 is a perspective view of a clamp and a flexible tube according to the invention;

FIG. 4 is a perspective view of a clamp according to the invention;

FIG. 5 is a perspective view of a clamp according to the invention;

FIG. 6 is a perspective view of the a clamp in clamping engagement with flexible tubing;

FIG. 7 is a view of another embodiment of a clamp of the invention;

FIG. 8 is a further view of the clamp of FIG. 7;

FIG. 9 is a side view of the clamp of FIG. 7 shown clamped closed without flexible tubing;

FIG. 10 is a further view of the clamp of FIG. 7 shown clamped closed without flexible tubing;

FIG. 11 is a further view of the clamp of FIG. 7;

FIG. 12 is a further view of the clamp of FIG. 7;

FIG. 13 is a view of another embodiment of a clamp of the invention shown open;

FIG. 14 is a view of the clamp of FIG. 13 shown closed;

FIG. 15 is a view of another embodiment of a clamp of the invention shown open;

FIG. 16 is a view of the clamp of FIG. 15 shown closed;

FIG. 17 is a view of another embodiment of a clamp of the invention shown open;

FIG. 18 is a view of the clamp of FIG. 15 shown closed;

FIG. 19 is a view of another embodiment of a clamp of the invention shown open;

FIG. 20 is a view of another embodiment of a lamp of the invention;

FIG. 21 is a cut-out view of the clamp of FIG. 20; and

FIG. 22 is a further cut-out view of the clamp of FIG. 20.

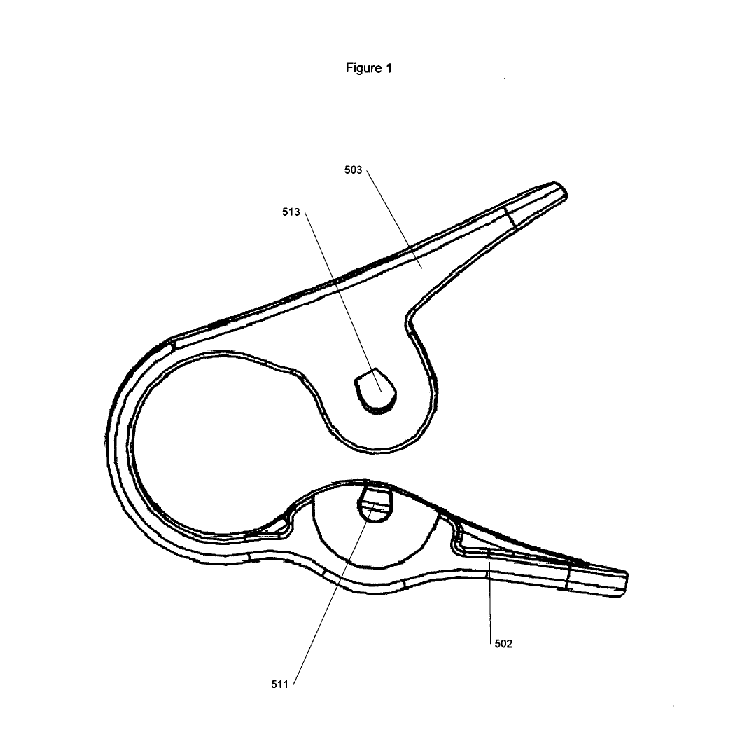

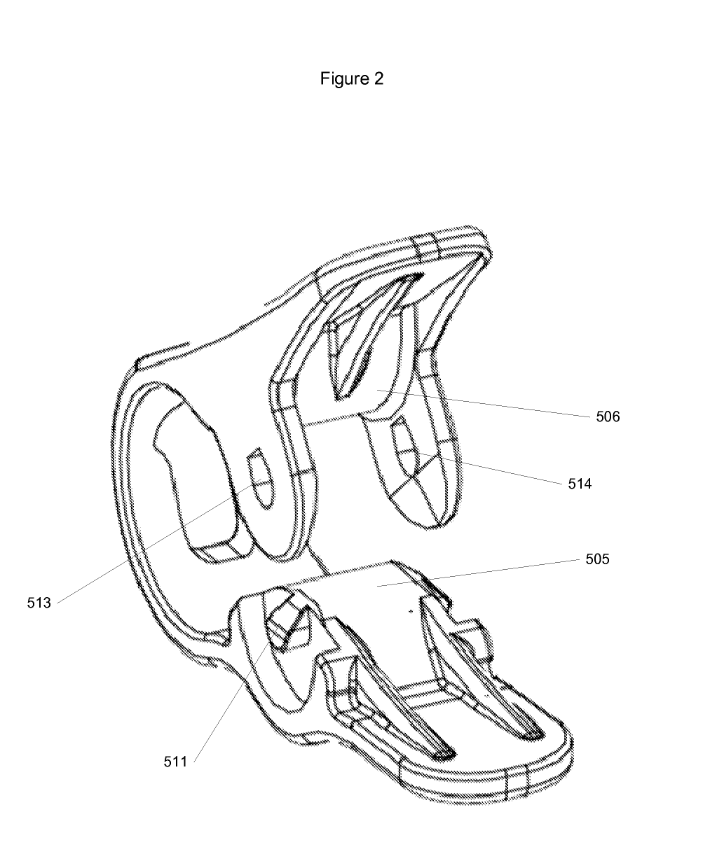

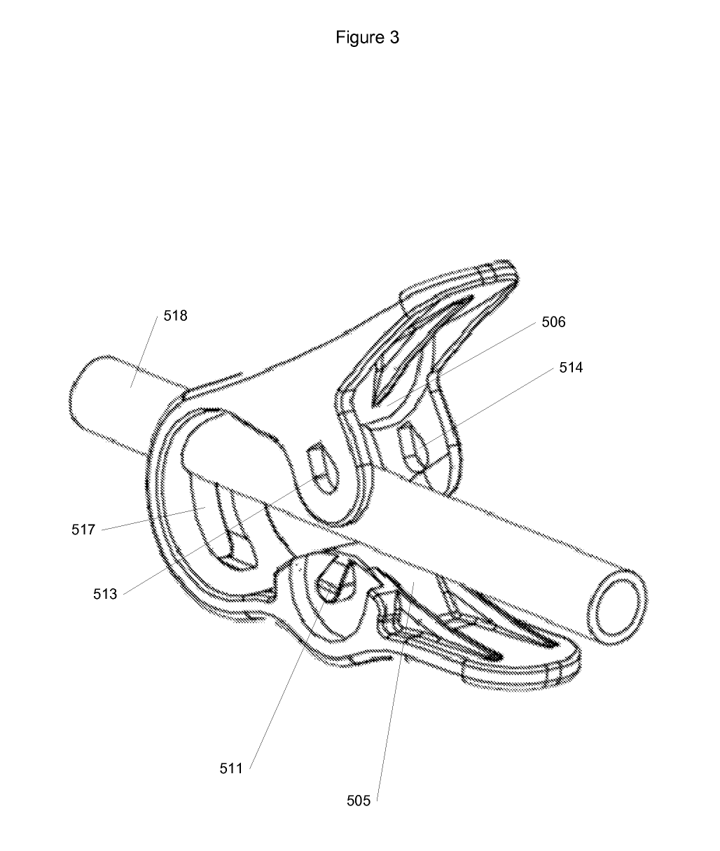

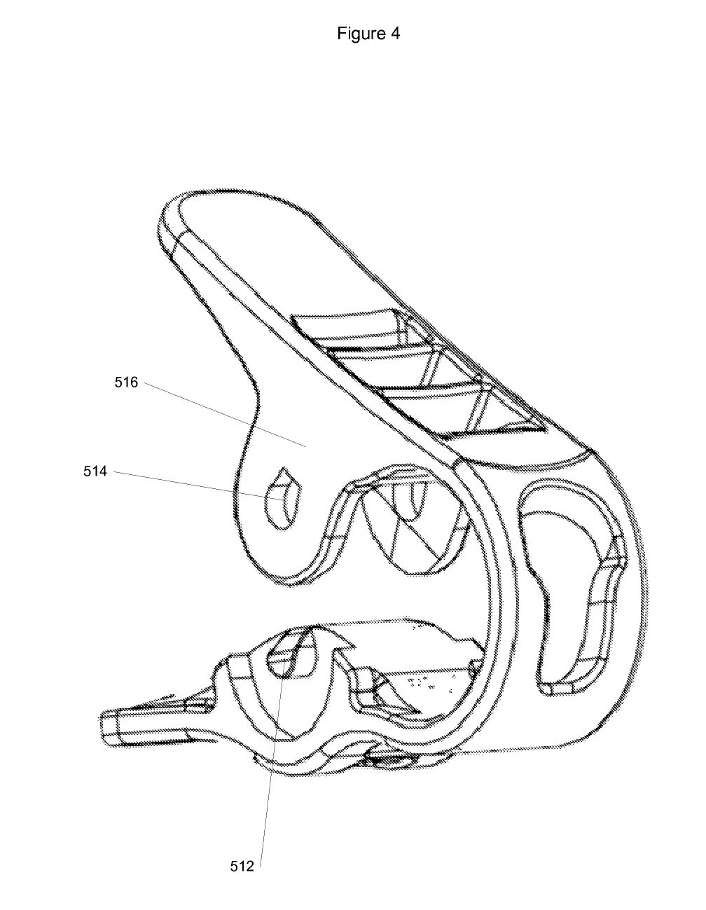

### PREFERRED EMBODIMENT OF THE INVENTION

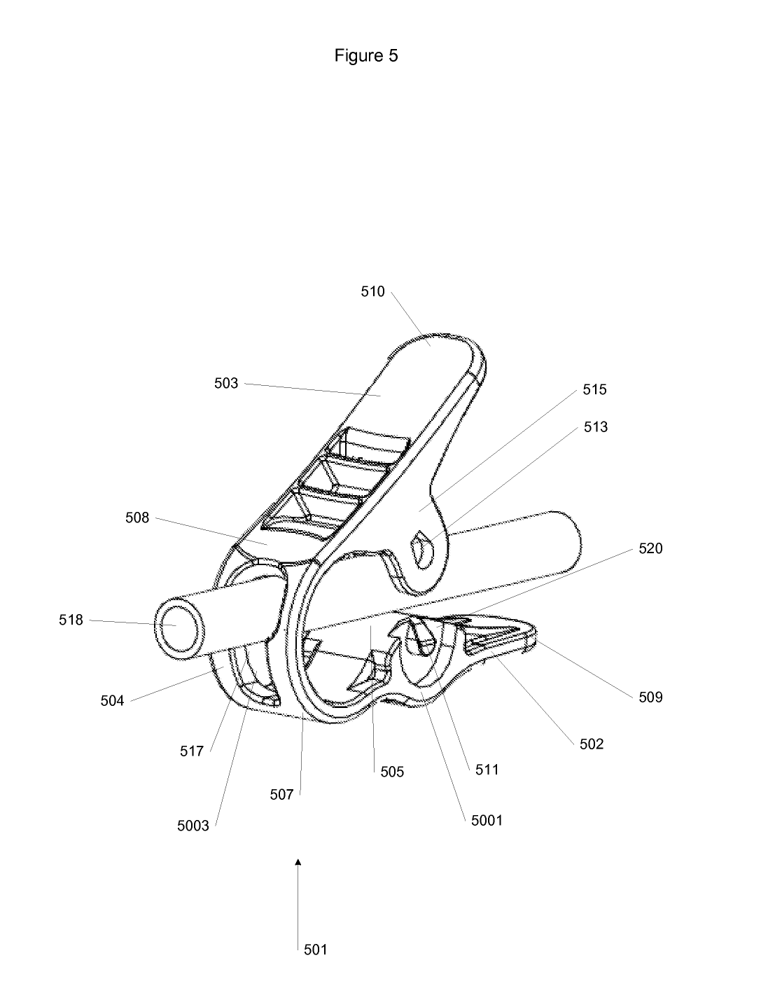

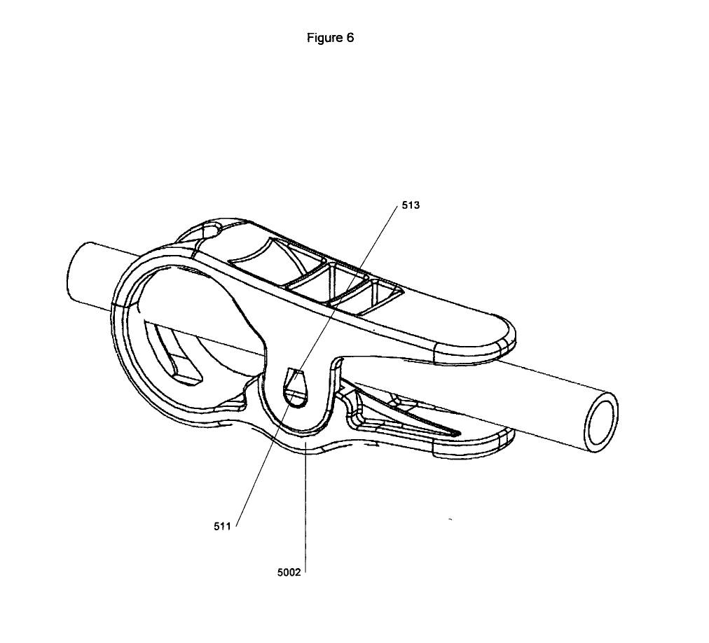

Referring initially to FIG. 5 there is shown a clamp 501 including two arms 502 and 503 hingedly connected by hinge portion 504. Each arm 502 and 503 has a clamping portion 505 and 506 such that relative rotation of the arms brings the clamping portions into clamping engagement.

The arms are hingedly connected at one end 507 and 508, the other end of each arm 509 and 510 each having a handle adapted for finger engagement. This provides leverage to allow a user to more easily close the clamp.

The clamping portion 505 and 506 of each arm 502 and 503 is located intermediate the ends of each respective arm. It would be understood that the clamping portion can be located at any suitable portion of the arms according to the particular requirement.

The clamp includes a lock 520 to keep the clamping portions in clamping engagement. In the preferred embodiment the lock is a non-reopening lock, however, in some embodiments the lock is a reopening lock that opens upon actuation by the user.

In this embodiment the lock 520 is located intermediate the ends of each arm. The lock includes locking tabs 511 and 512 on one of the arms 502 and receiving apertures or slots 513 and 514 on the other arm 503 for receiving the locking tabs, the locking tabs moving into the receiving slots when the clamping portions come into clamping engagement thereby locking the clamp. As would be understood the locking apertures or slots can be any suitable shape.

The locking tabs extend substantially perpendicular to the direction of relative rotation of the arms to ensure secure locking of the clamp. It would be understood that this provides secure locking of the clamp as the locking tabs act with a force perpendicular to the force that a user would normally exert on the clamp when locking the clamp. The locking tabs can, in other embodiments, extend in direction not substantially perpendicular to the direction of the relative rotation of the arms however this may provide for a less secure lock.

The locking tabs 511 and 512 and the receiving slots 513 and 514 are adapted for permanent engagement once the receiving slots receive the locking tabs thereby forming the non-reopening lock.

The other arm 503 includes shields 515 and 516 that extend substantially perpendicular to the direction of relative rotation of the arms, the shield having a hole therein to form the receiving slots 513 and 514. The shields are adapted to prevent finger engagement by a user thereby substantially preventing the user from unlocking the clamp. Parts 5001 and 5002 also act to prevent finger engagement by the user. The build up area on the one arm 502 also prevent finger engagement by the user.

The clamp includes an aperture 517 through which a flexible length of tubing 518 is guided. The tubing then runs along the length of the clamp from the aperture 517, between the clamping portions 505 and 506 and between the two end of the clamp 509 and 519. The tubing is held in place by the aperture 517 and also by the shields 515 and 516 which also act as a guide for the tubing to prevent it from slipping out of a clamping position in which it will be clamped shut when the clamp is sealed and the clamping portions engage it. In one embodiment, the flexible tubing is fed through the aperture by a health care worker prior to inserting one end of the tubing into a bag for receiving blood (or a bag for dispensing an IV liquid). In other embodiments, the flexible tubing is fed through the aperture at an assembly point and the ends of the flexible tubing are permanently connected to other components at its ends. Components that may be connected to the ends of the tubing are sampling pouches, bags for other blood components, multisampling luer adaptors, sampling ports, connectors, needles, bloodbags or other components as required. This allows a single unit with the clamp already installed to be provided to a user.

In use, health care worker withdraws blood from a donor or patient, or inserts an IV into a patient using a needle which is located at one end of the flexible tubing. The other end of the tubing is connected to an empty bag to receive blood donations or a bag filled with IV liquid in the event that the patient is being treated using the liquid. Once the blood is collected or the IV liquid has been drained, the health care worker engages the lock by placing their thumb and forefinger, one each on the outer side of the two arms 502 and 503, engaging the handles located at the ends 509 and 510 of the arms. The user then applies pressure to squeeze the arms of the lock together, thereby moving the locking tabs 511 and 512 into locking engagement with the receiving slots 513 and 514 thereby locking the lock. The flexible tubing, being located between the clamping portions 505 and 506 is then clamped shut preventing the flow or blood or any other liquid along the tubing. The tubing is therefore effectively sealed to prevent spills or contamination of the collected sample. The clamp includes means for preventing the clamp from sliding freely along the length of tubing. This is advantageous as it facilitates the manufacturing process as the clamp can be wedged to a predefined section of the tubing. As shown in FIG. 5, in the preferred embodiment the means for preventing the clamp from sliding along the length of the tubing is provided by a narrowing 5003 of the aperture 517.

In this embodiment, the clamping portions 505 and 506 are substantially C shaped and extend along the majority of the width of the clamps. This ensures that a piece of flexible tubing placed in the clamp will be securely clamped even if it moves laterally between the two sides of the clamp. The shields also assist in maintaining the flexible tubing in a suitable clamping position. In this embodiment the locking tabs 511 and 512 each extend from opposite sides of the clamping portion 505. This is preferred as this ensures that the locking mechanism is directly adjacent and in line with the clamping mechanism. This assists in keeping the flexible tubing securely clamped as even if the locking tab moves slightly within the receiving slots, the effect of the clamping portions on the flexible tubing will be negligible and the flexible tubing will remain securely clamped.

As can be seen, the locking tabs and receiving slots are in line (with reference to the length of the arms of the clamp) with the clamping portions. While this is preferred, the alignment of the clamping portions and the locking tabs and receiving slots can be moved as required. That is, these parts of the clamp do not have to be in alignment.

It would be understood that other embodiment of the invention can have differently shaped clamping portions. Some embodiments include angled edges, triangular shaped clamping portions, square clamping portions and any other suitable shape. It would be understood that C shaped, or other curve shaped clamping portions are preferred as they provide a larger clamping area and provide room for movement if the locking mechanism moves slightly.

The preferred embodiment of the invention is made of a single piece of plastics material. The material chosen is flexible enough to allow the hinge portion 504 to move flex sufficiently so as not to break when the clamp is being closed but hard enough to ensure that the clamp is sufficiently rigid to support the non-reopening action and the clamping action. It would be understood that the clamp can be made of multiple sections that are fitted together. The fitting may include providing snap locking parts that are mutually engageable. In one embodiment the arms are provided separately and then fitted together by a pin hinge, each arm having a suitable hinge joint for receiving the hinge pin. It will also be understood that the clamp can be made of any suitable material other than plastics material and the material may be chosen according to the particular requirement.

In one preferred embodiment, the clamp is made of a single piece of plastics material through a process of plastic injection molding. This process uses plastic chips or pellets which are usually dried in a hopper before going to an extrusion feed screw. The plastics material is then heated to molten state by a combination of heating elements and shear heating from the extrusion screw. The screw and hydraulic motion forces the resin into a mold forming the desired shape, and the mold is cooled to a specific temperature to allow solidification of the resin. The mold is then opened and the parts ejected.

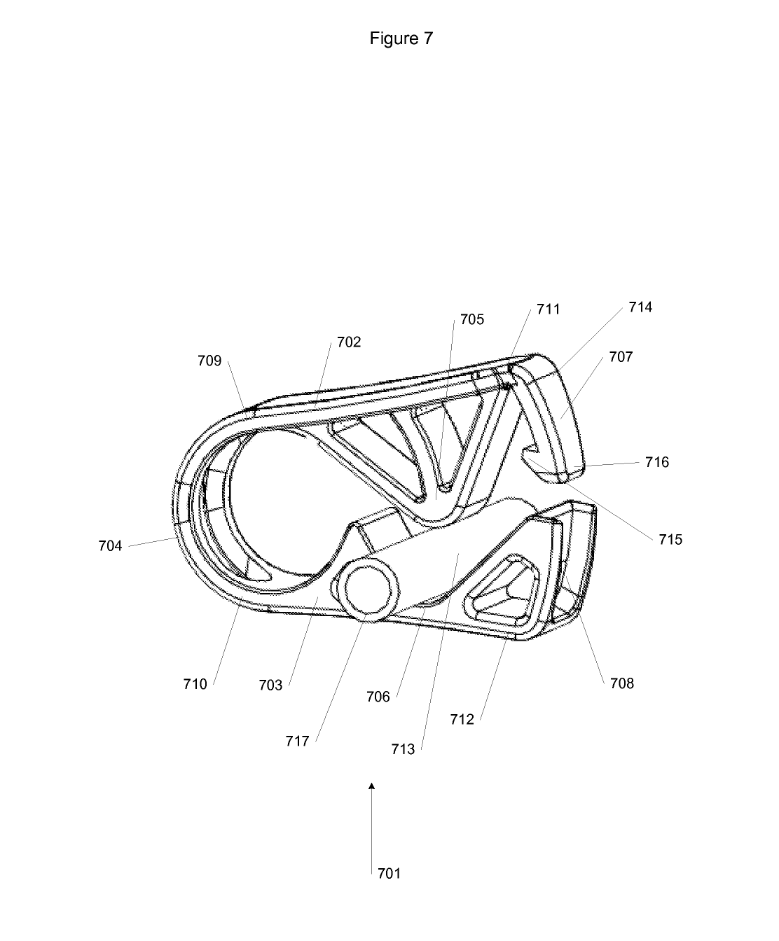

A further embodiment of a clamp is shown in FIGS. 7 to 12. This clamp is designed for applications where the tubing ends are not available for insertion into the slot in the clamp end. This need arises at the end-user where they have a requirement for shutting off certain tubes at certain stages of the process and in that case the ends of the tubing are either pre-connected to bags or to other devices like needles or sampling ports.

As can be seen the top section of the clamp which has a hook flexes over the bottom section and clips into the slot/opening in the bottom section. The clamp has been designed to be applied to tubing without the need for the tubing to be inserted into the clamp.

Referring now to FIG. 7 there is shown a further embodiment of a clamp according to the invention. Clamp 701 has two arms 702 and 703 hingedly connected by hinge portion 704. Each arm has a clamping portion 705 and 706 such that relative rotation of the arms brings the clamping portions into clamping engagement.

One of the arms 702 has a locking tooth 707 and the other arm 702 has a receiving slot 708 for receiving the locking tooth, the locking tooth moving into locking engagement with the receiving slot when the clamping portions come into clamping engagement thereby locking the clamp.

The arms are hingedly connected at their proximal ends 709 and 710 by the hinge portion 704.

The locking tooth 707 is located at the distal end 711 of the one arm 702 and the receiving slot is located at the distal end 712 of the other arm 703.

As can be seem from the Figures, the clamping portions are preferably located intermediate the ends of each respective arm. The position of the clamping portions can be adjusted according to the requirements of the particular application for which the clamp is designed.

The clamp has a clamping zone 713 in which objects to be clamped are clamped when the clamping portions are brought into clamping engagement. In this embodiment the clamping zone is adapted to receive a length of flexible tubing however the size and shape of the clamping zone can be adjusted according to the particular requirements of the application of the clamp. In this embodiment the clamping zone is adapted to receive a length of flexible tubing 717, the length of flexible tubing running substantially perpendicular to the length of the clamp. It would be understood that the flow of liquid through the tube is also substantially perpendicular to the length of the clamp and that the clamping portions apply sufficient clamping pressure along a cross section of the flexible tubing placed in the clamping zone such that the flow of liquid in the flexible tubing is stopped.

In this embodiment one of the clamping portions 705 is triangular shaped and the other clamping portion 706 is adapted to receive the triangular shaped clamping portion. It would be understood that the shapes of the clamping portions can take any suitable shape and that particular shapes may be more advantageous for particular applications.

The locking tooth 707 is connected at one end 714 to the distal end 711 of the one arm 702 and has an abutment locking tab 715 at its other end 716. The receiving slot 708 is adapted to receive the abutment locking tab to thereby lock the clamp in clamping engagement.

In this embodiment the locking tooth is adapted to be permanently engagable with the receiving slot such that the clamp is substantially a non-reopening clamp. That is, in one application it is preferable that a user cannot easily re-open the lock without breaking or damaging the clamp. In some other embodiments the clamp is a re-opening clamp that can be opened and closed multiple times.

The arms 702 and 702 are adapted for finger engagement by a user. Suitable rubber grips, ridges or other suitable means to allow easy finger engagement are provided on the arms is required.

FIG. 8 shows another view of the clamp of FIG. 7. This view shows the clamping portions 705 and 706 coming together ready to clampingly engage the flexible tubing 717 which is in the clamping zone 713. To further engage the clamp into clamping engagement a user uses their thumb and forefinger to grip the arms 702 and 703 and squeeze the arms together applying a compressing force and causing the locking tooth 707 to locking engage with the receiving locking slot 708 thereby locking the clamp. When the clamp is locked the flexible tubing is clamped together preventing the flow of liquid past the clamping zone.

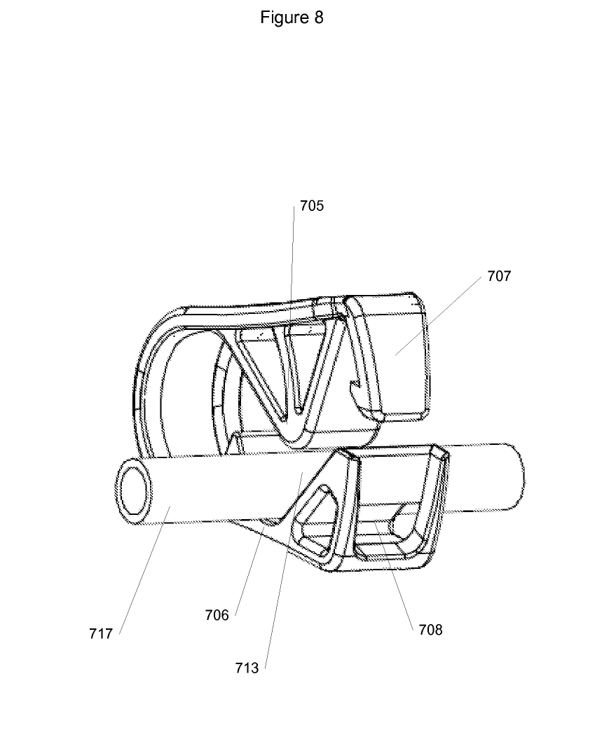

FIG. 9 shows the clamp in clamping engagement. It can be seen that the clamping portions 705 and 706 are mutually engageable to form an effective clamping zone 713.

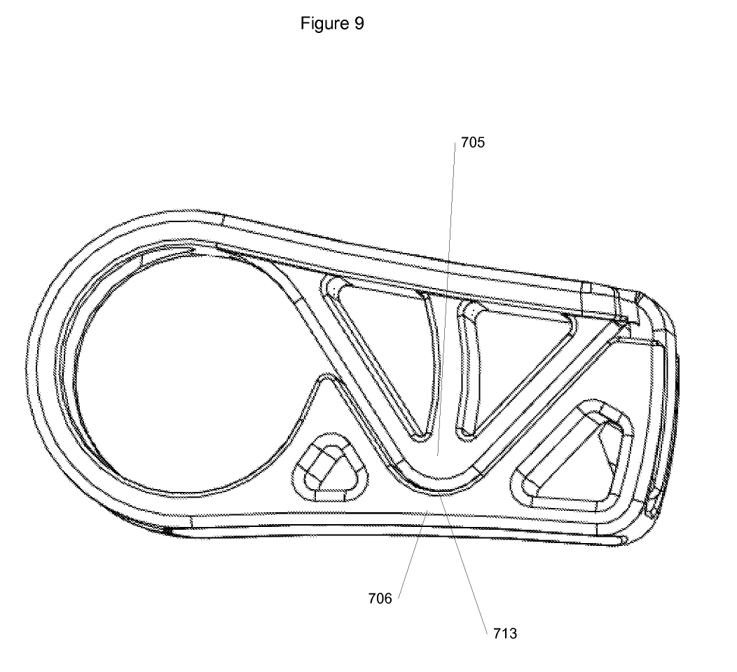

FIG. 10 shows another view of the clamp in clamping engagement and shows the locking tooth 707 in locking engagement with the receiving locking slot 708. The clamp also includes support struts such as strut 718 to provide support for the structure of the clamp. Parts 1001 and 1002 prevent finger access to the locking tooth 707 by a user. It would be understood that any suitable lock can be used and the locking mechanism need not be a locking tooth but can be any suitable lock or any of the locking mechanism as described herein.

FIG. 11 shows another view of the clamp of FIG. 7. This view shows the clamping portions 705 and 706 coming together ready to clampingly engage the flexible tubing 717 which is in the clamping zone 713.

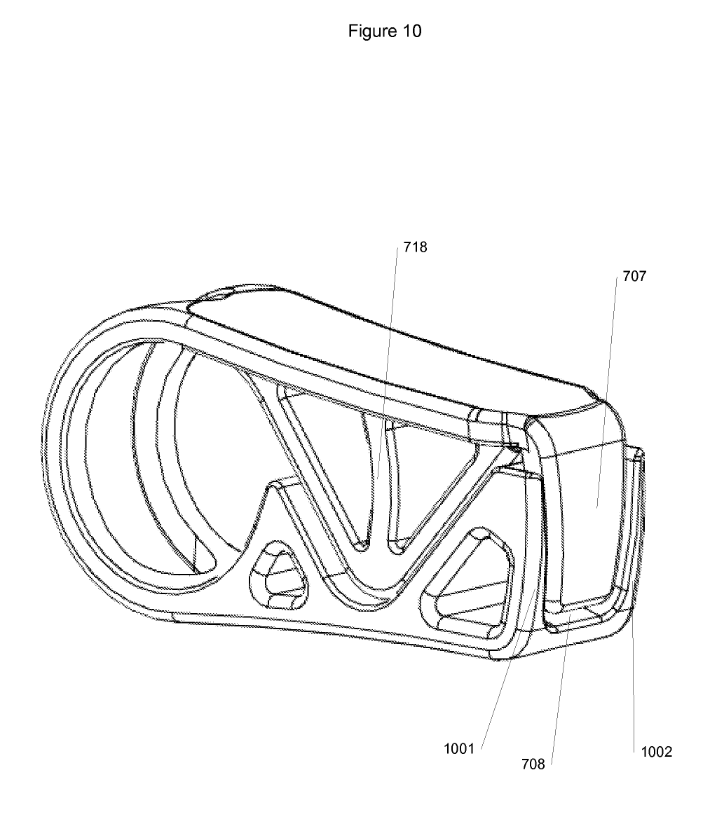

FIG. 12 shows another view of the clamp of FIG. 7. This view shows the clamping portions 705 and 706 coming together ready to clampingly engage the flexible tubing 717 which is in the clamping zone 713.

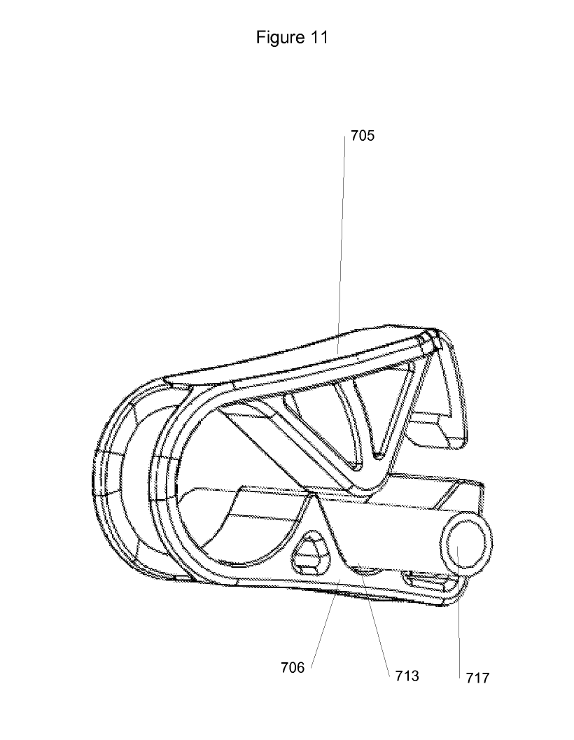

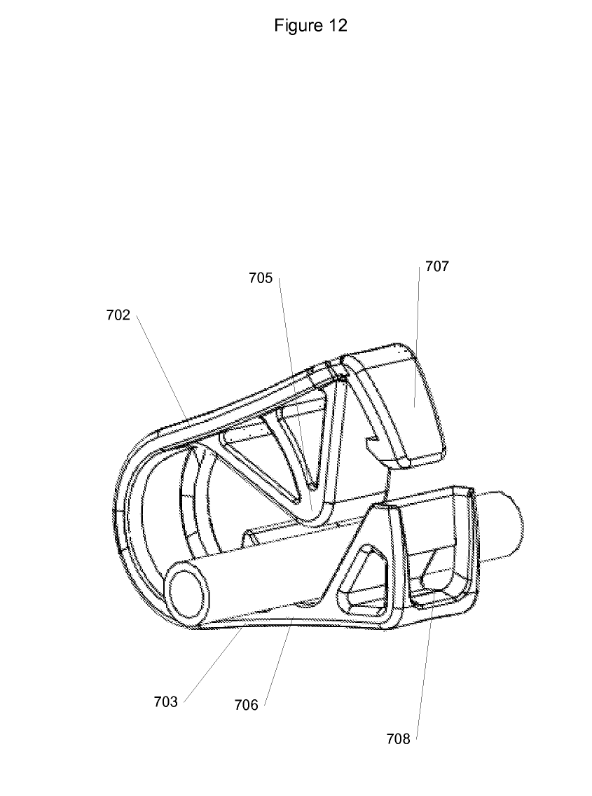

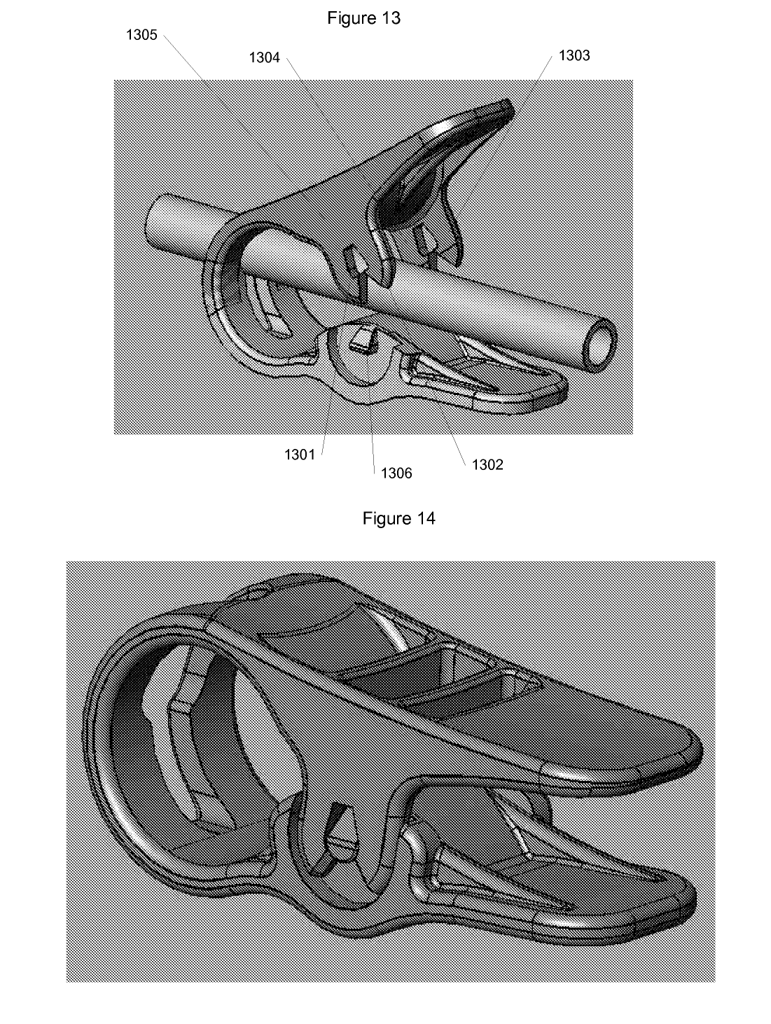

FIGS. 13 and 14 show another embodiment of the invention where the locking mechanism includes a first latch having parts 1301 and 1302 and a second latch 1303 and 1304 on arm 1305. When the clamp is closed upon compression by a user, the latch parts are pushed apart by the locking tabs 1306 (only one side shown) until the locking tab is encompassed by the latches which then clampingly engage the locking tab thereby locking the clamp as shown in FIG. 14.

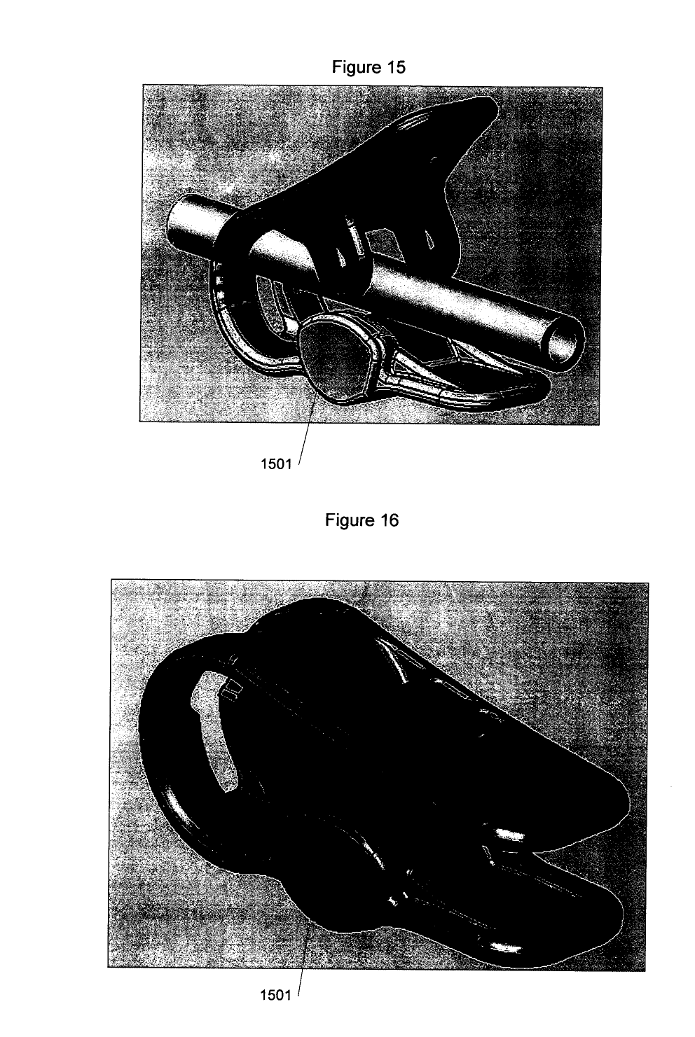

FIGS. 15 and 16 show another embodiment of the invention where the locking tabs are enclosed by casings or shrouds 1501 (only one side shown). The casing prevents users from being able to engage the locking mechanism once the lock is closed and the clamp closed in clamping engagement.

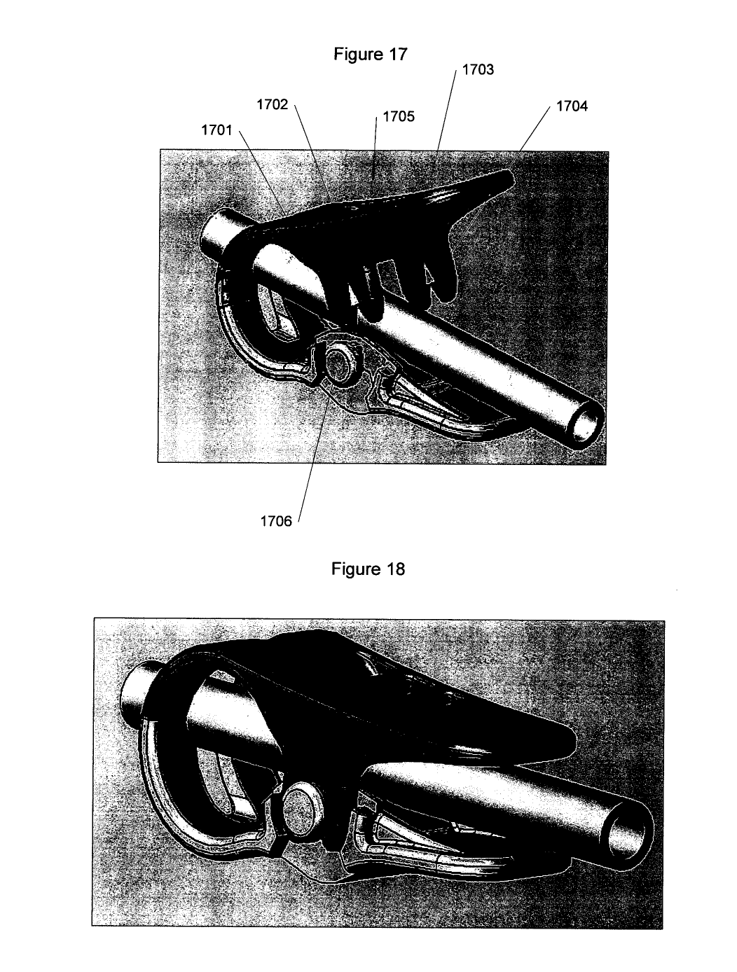

FIGS. 17 and 18 show another embodiment of the invention where the locking mechanism includes a first latch having parts 1701 and 1702 and a second latch 1703 and 1704 on arm 1705. The locking tabs 1706 (only one side shown) are mushroom shaped to prevent a user being able to engage the tabs. When the clamp is closed upon compression by a user, the latch parts are pushed apart by the locking tabs 1706 (only one side shown) until the locking tab is encompassed by the latches which then clampingly engage the locking tab thereby locking the clamp as shown in FIG. 18.

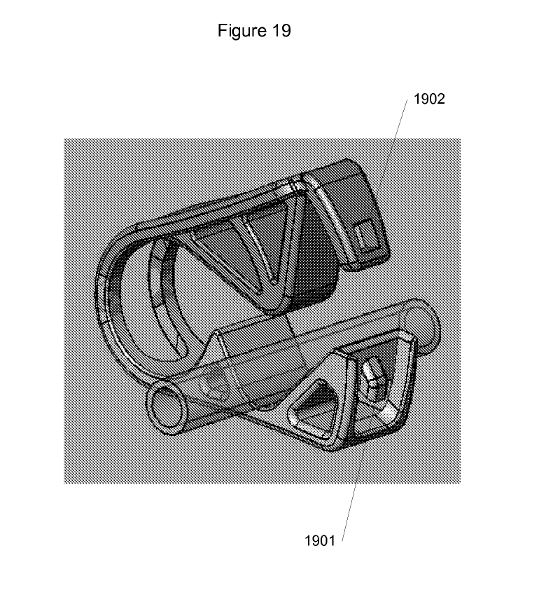

FIG. 19 shows another embodiment of the invention that includes a locking mechanism having a locking pip 1901 and locking aperture 1902.

In use, the user takes the clamp in one hand and the tubing to be clamped in the other hand. The tubing is then guided into the clamping zone by the user and aligned so that it is ready to be clamped. The user then applies a compression to both arms of the clamp using their thumb and forefinger to bring the arms and the clamping portions together. The clamping portions then compress and clamp the flexible tubing preventing flow of the liquid through the flexible tubing. The locking tooth engages and locks into the receiving locking slot thereby locking the clamp and allowing the user to remove the compression force.

The clamps are preferably a unitary plastic injection molding of a flexible and resilient polymeric material such as polypropylene or PVC. In some embodiments the clamp is designed to produce and audible click once locked. This provides the user with confirmation that the clamp is securely locked and that the user can stop applying the compression force.

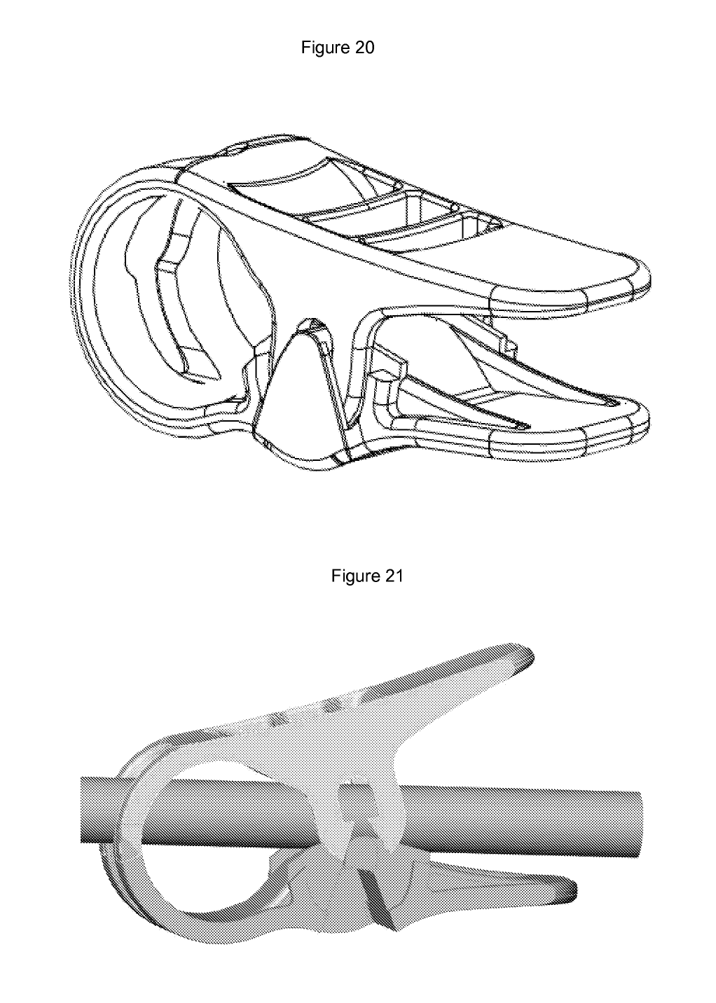

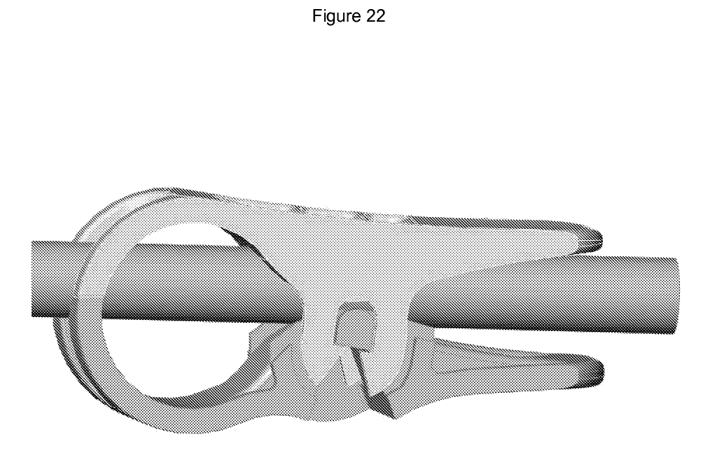

FIG. 20 shows a variation which replaces the slots with two tabs (arrow head shaped). FIGS. 21 and 22 show the “cut-out” version to show the arrow head tabs. It can be seen in FIG. 20 that when engaged the tabs cannot be dislodged and unlocked. This physically stops the tabs from being dislodged from the locking position. As would be understood, any locking mechanism such as slots and engaging tabs or tabs/arrow heads engaging slots such as those shown in FIGS. 20 to 22 may be used. It is preferred that whatever type of locking mechanism is used offers a non reopening type lock for the reasons identified above. It is preferable in any locking mechanism chosen that the separation forces act perpendicular to the locking mechanism as this secures the clamp and prevents is from being accidentally or even intentionally opened.

As discussed above, the present invention discloses an embodiment that has an aperture that allows for tubing to be threaded into the open clamp to allow assembly to the tubing (eg. by the manufacturer of the blood bags) prior to connecting the tubing ends to other components like bloodbag, needle hubs, luer connectors, etc. This embodiment advantageous is when the bag manufacturer provides the clamp already preassembled to the specific portion of tubing where the clamp is eventually required to be closed by the end-user.

A second preferred embodiment is designed not to be pre assembled to tubing, but allows the clamp to be applied or closed over (across) the tubing where the tubing ends are not amenable for threading the clamp (generally because the ends have other components already attached). This embodiment is for the end-user to apply and cannot be threaded by tubing.

Although the invention has been described with reference to specific examples, it will be appreciated by those skilled in the art that the invention may be embodied in many other forms.

1. A clamp including:
two arms hingedly connected;
each arm having a clamping portion such that relative rotation of the arms brings the clamping portions into clamping engagement, wherein one of the arms includes a plurality of locking tabs and the other arm includes a plurality of receiving apertures for receiving the locking tabs, the locking tabs moving into the receiving apertures when the clamping portions come into clamping engagement thereby locking the clamp.

2. The clamp according to claim 1 wherein the arms are hingedly connected at one end, the other end of each arm having a handle adapted for finger engagement.

3. The clamp according to claim 2 wherein the clamping portion of each arm is located intermediate the ends of each respective arm.

4. The clamp according to claim 3 wherein the clamp includes a lock to keep the clamping portions in clamping engagement.

5. The clamp according to claim 4 wherein the lock is a non-reopening lock.

6. The clamp according to claim 5 wherein the lock is located intermediate the ends of each arm.

7. The clamp according to claim 6 wherein the locking tab extends substantially perpendicular to the direction of relative rotation of the arms.

8. The clamp according to claim 7 wherein the one arm includes two locking tabs each locking tab extending substantially perpendicular to the direction of relative rotation of the arms and the other arm includes two receiving apertures for receiving a respective locking tab.

9. The clamp according to claim 8 wherein the locking tabs and the receiving apertures are adapted for permanent engagement once the receiving apertures receive the locking tabs thereby forming the non-reopening lock.

10. (canceled)

11. The clamp according to claim 9 wherein the other arm includes a shield extending substantially perpendicular to the direction of relative rotation of the arms, the shield having a hole therein to form the receiving aperture.

12. The clamp according to claim 11 wherein the other arm includes two shields extending substantially perpendicular to the direction of relative rotation of the arms, each shield having a hole therein to form the receiving apertures.

13. The clamp according to claim 12 wherein the shields are adapted to prevent finger engagement by a user thereby substantially preventing the user from unlocking the clamp.

14. The clamp according to claim 13 wherein the clamp includes an aperture through which a flexible length of tubing is guided.

15. A clamp including:
two arms hingedly connected;
each arm having a clamping portion such that relative rotation of the arms brings the clamping portions into clamping engagement;
one of the arms having a locking tooth and the other arm having a receiving aperture for receiving the locking tooth, the locking tooth moving into locking engagement with the receiving aperture when the clamping portions come into clamping engagement thereby locking the clamp.

16. (canceled)

17. (canceled)

18. (canceled)

19. (canceled)

20. A clamp according to claim 15 wherein the clamp has a clamping zone in which objects to be clamped are clamped when the clamping portions are brought into clamping engagement and the clamping zone is adapted to receive a length of flexible tubing.

21. (canceled)

22. (canceled)

23. (canceled)

24. A clamp according to claim 15 wherein the locking tooth is connected at one end to the distal end of the one arm and has an abutment locking tab at its other end.

25. A clamp according to claim 24 wherein the receiving aperture is adapted to receive the abutment locking tab to thereby lock the clamp in clamping engagement.

26. A clamp according to claim 25 wherein the locking tooth is adapted to be permanently engagable with the receiving aperture such that the clamp is substantially a non-reopening clamp.

27. (canceled)

28. (canceled)

29. A clamp including:
two arms hingedly connected;
each arm having a clamping portion such that relative rotation of the arms brings the clamping portions into clamping engagement, wherein the arms includes a locking mechanism for locking the clamping portions together when the clamping portions come into clamping engagement thereby locking the clamp.

30. A clamp according to claim 29 wherein the locking mechanism is a non-reopening locking mechanism.
